(19) United States
(12) Patent Application Publication
(10) Pub. No.: US 2006/0142254 A1
(43) Pub. Date: Jun. 29, 2006

(54) Antipyretic preparation containing xylitol

(75) Inventors: Daniel Wils, Morbecque (FR); Roselyne Bernard, Lestrem (FR)

Correspondence Address: YOUNG & THOMPSON, 745 SOUTH 23RD STREET, 2ND FLOOR, ARLINGTON, VA, 22202, US

(73) Assignee: ROQUETTE FRERES, Lestrem (FR)

(21) Appl. No.: 11/360,594

(22) Filed: Feb. 24, 2006

Related U.S. Application Data
(62) Division of application No. 10/314,515, filed on Dec. 9, 2002.

(30) Foreign Application Priority Data
Dec. 10, 2001 (FR) 01 15917

(51) Int. Cl. A61K 31/60; A61K 31/045

(52) U.S. Cl. 514/165; 514/738

(57) ABSTRACT

The invention relates to an antipyretic preparation intended to be administered by any means other than orally, characterized in that it comprises xylitol, and more particularly to an antipyretic preparation characterized in that it consists of an antipyretic and a synergistic amount of xylitol. The invention also relates to the use of this antipyretic preparation in pharmaceutical applications, in which the xylitol can then be used as a medicinal product with an antipyretic effect, for example in combination with another medicinal product of the family of antipyretic analgesics which may or may not have anti-inflammatory action. It also relates to its use in the treatment of infections chosen from the group of parasitic, viral and bacterial infections, but also in the treatment of heatstroke, burns, poisoning with a chemical product or any disturbance of the function of body temperature regulation.

This application is a division of co-pending application Ser. No. 10/314,515, filed on Dec. 9, 2002, the entire contents of which are hereby incorporated by reference.

The present invention relates to an antipyretic preparation.

The invention also relates to an antipyretic preparation comprising xylitol, intended to be administered by any means other than orally.

More particularly, the invention relates to an antipyretic preparation consisting of an antipyretic and a synergistic amount of xylitol, this preparation being intended to be administered to humans or animals by any means other than orally.

For the purpose of the invention, the term “antipyretic” or “antipyretic agent” is intended to mean a compound which has the property of lowering body temperature in humans or in animals, when this temperature is at an abnormally high level compared to physiological values for humans or the animal.

For the purpose of the invention, in addition, the term “effective amount” of xylitol is intended to mean an amount of xylitol in which, when acting synergistically with an antipyretic, makes it possible to optimise the lowering of body temperature.

Many substances have been known for a long time for their pyrogenic effects, in the broad sense, in humans and animals. These are sulphur, milk, certain gums, certain steroidal hormones, 2,4-dinitrophenol, tetra-hydro-β-naphthylamine and carmine.

The metabolites synthesized by certain micro-organisms have also been known for a long time for their pyrogenic properties, such as the endotoxins of Gram-negative bacteria or the RNA of Candida utilis.

In response to the increase in temperature noted in humans or animals exposed to these agents, particular active principles which lower body temperature, active principles called antipyretics, are used.

To bring the body temperature back to its physiological value (for example 37° C. for humans), the organism increases its heat losses via mechanisms such as peripheral vasodilation, hypersudation and inhibition of prostaglandin synthesis, in the thermoregulatory centres.

To lower the body temperature, antipyretic analgesics with notable anti-inflammatory action, such as aspirin and derivatives thereof, and antipyretic analgesics virtually lacking anti-inflammatory properties, such as para-aminophenol derivatives (such as paracetamol), pyrazolone derivatives and various medicinal combinations with antipyretic analgesics, are conventionally used.

Those skilled in the art readily find the methods of administration and the doses of all these antipyretics in the VIDAL dictionary.

It should be noted, however, that besides the development of these substances with antipyretic effects, a certain number of studies integrating polyols have also been undertaken.

Specifically, besides their properties as texturing agents with a sweet flavour and their notable chemical stability, it is especially their properties of bacteriological stability and their non-fermentability which have led to the use of polyols not only in the food domain, but also in human therapy.

Thus, in the particular case of xylitol, besides its use as a sweetener for diabetics and as an acariogenic agent, it has also been described in a certain number of therapeutic applications, as illustrated by the teachings of the following patents:
in EP patent 150,053, xylitol is incorporated into weakly osmotic, hypocalorific aqueous preparations delivered by infusion to patients in an extremely serious condition, suffering from severe trauma or stress, or kidney disease, or placed under ventilation. Xylitol provides the only energy source of these preparations.
In U.S. Pat. No. 5,719,196, the xylitol is incorporated into oral preparations intended for treating, in humans, infections of the acute otitis type, severe respiratory infections, acute bronchitis, sinusitis and conjunctivitis. The xylitol, in solid form, is administered at 6 to 10 g per day, and it is described as an inhibitor of growth of the pathogenic micro-organisms responsible for these diverse infections.

Xylitol is, finally, used in many other medical applications, such as preventing adrenocortical suppression during steroid therapy, improving haemolysis induced by certain drugs, reducing liver and bile disfunctions, etc., as illustrated in the article by K. K. MÄKINEN in Medical Hypotheses (2000), 54-4, pp. 603-613.

To the applicant company's knowledge, however, only the tests undertaken by A. SHIMOHIRA, reported in TOKYO TORITSU EISEI KENKYUSHO KENKYU NEMPO in 1969 (volume 1971 pp. 83-88) refer to body temperature-lowering effects in relation to the intravenous administration of xylitol.

This involved determining the decrease in body temperature of a laboratory animal, in this case a healthy rabbit, subjected to the action of various molecules administered intravenously.

Xylitol was thus tested along with molecules such as ethylephedrine and methylephedrine.

The results indicated that xylitol, just like methylephedrine, lowers the body temperature of the laboratory animal.

This study, carried out in animals in good health, teaches that xylitol activates the “thermolysis” phenomena which lead to lowering of body temperature in animals.

However, the teaching which those skilled in the art can draw from this observation is especially the fact that xylitol, in laboratory animals and, a fortiori, in humans, has a temperature-lowering effect in healthy individuals.

The applicant company has, however, to its credit, taken up the studies on xylitol, with the aim of developing preparations capable of decreasing body temperature, not for healthy individuals, but in cases of fever induced during an infectious process.

Specifically, it is known by those skilled in the art that the temperature-lowering mechanisms of an individual who is ill differ fundamentally from those which direct body temperature lowering in a healthy individual.

To the applicant company's knowledge, it is not described or suggested anywhere in the state of the art that xylitol may have the same effect in these two physiological problems which are so dissimilar.

After many investigations, the applicant company has thus shown that xylitol can be administered, by any method other than orally, with an antipyretic when there is an increase in temperature resulting from a stimulation of the immune system, from an infection or from an inflammatory process, which are phenomena involving physiological mechanisms other than thermogenesis.

More particularly, the applicant company has succeeded in developing, at the cost of much research, antipyretic preparations intended to be administered by any means other than orally, these preparations consisting of xylitol or of an antipyretic and a synergistic amount of xylitol.

The applicant company in fact has a unit for production of apyrogenic xylitol, the intended applications of which are those mentioned above.

The applicant company has therefore undertaken, on laboratory animals exhibiting hypothermia, comparative tests aimed at studying the body temperature-lowering effects of a medicinal product known for its antipyretic properties, compared with xylitol taken alone or in combination.

Entirely unexpectedly, given the complex and multifactorial nature of the physiological mechanisms developed in the organism to lower the body temperature of an animal in good health or, on the other hand, of an animal in a state of hypothermia, the applicant company has shown that it is possible, using xylitol, not only to notably lower the body temperature of sick rabbits, but also to potentiate, through xylitol, the temperature-lowering effects of conventional antipyretics.

The supplementary effect provided by the xylitol is not therefore an additional effect of two different antipyretic agents, but clearly a combination of effects, the temperature difference noted being much greater than the temperature difference caused by each of the two components of the preparation administered when taken separately, as will be exemplified below.

The applicant company has therefore found that this effect can be provided by preparing an antipyretic preparation intended to be administered by any means other than orally, characterized in that it comprises xylitol.

Surprisingly, and unexpectedly, the applicant company has also noted that this antipyretic effect can be increased with a preparation consisting of an antipyretic and a synergistic amount of xylitol.

The applicant company has found, moreover, that this preparation can be administered via a route chosen from the group consisting of intravenous, intramuscular, intraperitoneal and rectal administration.

The tests carried out in the rabbit, the reference animal chosen as a model for assessing hypothermias caused by certain molecules, make it possible to establish the proportions of xylitol and of antipyretic agents for demonstrating the effect of xylitol alone, or the synergistic effect when combined with an antipyretic agent.

Specifically, the pyrogen test in rabbits is the test described in the European Pharmacopoeia for verifying, in vivo, the apyrogenicity of certain molecules intended to be administered parenterally.

Thus, the applicant company, a producer of molecules intended to be administered parenterally, therefore used this test, which it has perfected, under the conditions described in the European Pharmacopoeia, in order to determine the body temperature-lowering effects of xylitol alone, and of xylitol combined with an antipyretic.

The applicant company has thus found that, in the antipyretic preparation intended to be administered other than orally in accordance with the invention, the xylitol content is between 0.5 and 15 g, these contents being understood as amount administerable per kg of body weight and per day.

Similarly, the applicant company has shown that this amount of xylitol, when it is combined with an amount of antipyretic of between 2 and 100 mg/kg of body weight, produces a significant increase in the effect of lowering the body temperature of the laboratory animal in a state of hypothermia, this lowering of the temperature being brought about by the antipyretic alone.

A compound of the family of antipyretic analgesics with anti-inflammatory action, of the aspirin family, in this case ASPEGIC®, was chosen as antipyretic substance.

The remarkable body temperature-lowering effects observed mean that the injectable preparations in accordance with the invention are thus intended for pharmaceutical applications, in which the xylitol can then be used as a medicinal product with antipyretic effect, for example in combination with another medicinal product of the family of antipyretic analgesics which may or may not have anti-inflammatory action.

It is also possible to use the preparation in accordance with the invention in the treatment of infections chosen from the group of parasitic, viral and bacterial infections, but also in the treatment of heatstroke, burns, poisoning with a chemical product, or any disturbance of the function of body temperature regulation.

Other characteristics and advantages of the invention will become apparent upon reading the example which follows. It is, however, given here only by way of non-limiting illustration.

### EXAMPLE 1

The experiment is carried out with batches of laboratory animals, in this instance here, rabbits.

The solutions to be injected are prepared in sterile water (water for injectable preparation—AGUETTANT).

The bottles of endotoxins (control standard Entotoxin or CSE—ENDOSAFE) contain 5 ml of endotoxins at 30 EU/ml with 120 ml of water for injectable preparation.

The contamination is calculated so as to observe a pyrogenic shock in the rabbit. This contamination is produced by intravenously injecting endotoxins in a proportion of 1.2 EU/ml, i.e. 12 EU/kg.

The antipyretic agent, in this instance aspirin, is prepared extemporaneously. 6.6 ml are added to 125 ml of endotoxins so as to obtain a dose equivalent to 100 mg/kg. The aspirin chosen is injectable ASPEGIC® (DL-lysine acetyl salicylate), manufactured by Laboratoires SYNTHELABO (LE PLESSIS ROBINSON), packed in proportion of 6 bottles of 2 g of powder and 6 vials of 5 ml of water for injectable preparation, for extemporaneous dissolution.

The xylitol is a batch of apyrogenic XYLISORB® manufactured by the applicant company. Apyrogenic dextrose monohydrate (marketed under the trademark LYCADEX® PF by the applicant company) is used as a control.

The rabbits are injected with a solution of xylitol at 150 g/l with water for injectable solution contaminated or not contaminated with the endotoxins. Five experiments are carried out with 3 rabbits per batch. Table I below gives the composition of the solutions injected into 5 batches of three rabbits.

TABLE I

| | | | Contaminated | |
|---|---|---|---|---|
| | | | water for | |
| Batch | | Product | injectable | |
| No. | Tests | injected | preparation | Aspirin |
| 1 | Negative | LYCADEX ® | No | No |
| | control | PF | | |
| 2 | Positive | LYCADEX ® | Yes | No |
| | control | PF | | |
| 3 | Aspirin | LYCADEX ® | Yes | Yes |
| | control | PF | | |
| 4 | Xylitol | Apyrogenic | Yes | No |
| | alone | XYLISORB | | |
| 5 | Xylitol + aspirin | Apyrogenic | Yes | Yes |
| | | XYLISORB | | |

The results of the measurements of the mean temperature differences carried out on the 5 batches of rabbits are given in Table II below.

TABLE II

| | | Difference compared with |
|---|---|---|
| | Temperature | the positive control of |
| | increase (° C.) | contamination (%) |
| Batch 1 | 0.35 | — |
| Batch 2 | 2.95 | 0 |
| Batch 3 | 1.57 | 46 |
| Batch 4 | 2.73 | 7.5 |
| Batch 5 | 0.82 | 72 |

A significant difference is established when the difference is greater than 5%.

It ensues that injection of xylitol already shows a significant effect, since a lowering of temperature of 7.5% is noted.

Next, the notable effect observed is that, when combined with aspirin, xylitol makes it possible here to lower by 72% the increase in temperature associated with injection of endotoxins, whereas aspirin alone, under the same operating conditions, only lowers the increase in temperature by 46%.

A synergy therefore clearly exists between xylitol (here at 1.5 g/kg) and ASPEGIC® (here at 100 mg/kg) injected intravenously, to activate thermolysis in rabbits having received a dose of endotoxins equal to 12 EU/kg.

This property of xylitol may therefore be exploited in the field of excipients for medicinal products for anipyretic purposes.

1. An antipyretic preparation comprising aspirin and a synergistic amount of xylitol, and in which the aspirin content is comprised between 2 and 100 mg and the xylitol content is comprised between 0.5 and 15 g, these contents being understood as amount administerable per kg of body weight and per day, as a medicinal product with an antipyretic effect, in combination with another medicinal product of the family of antipyretic analgesics with an inflammatory or anti-inflammatory action.

2. The preparation according to claim 1, wherein the preparation is in a form for intravenous, intramuscular, intraperitoneal and rectal administration.

3. The preparation according to claim 1, wherein said preparation is in the form of an injectable solution.

4. A method for treating infections selected from the group consisting of parasitic, viral and bacterial infections, heatstroke, burns, poisoning with a chemical product or any disturbance of the function of body temperature regulation, comprising administering an effective amount of the preparation according to claim 1 to said subject in need of treatment thereof by means other than orally to a human or warm blood animal.

5. The method according to claim 4, wherein the preparation is administered via a route selected from the group consisting of intravenous, intramuscular, intraperitoneal and rectal administration.

6. The method according to claim 4, wherein said preparation is administered intravenously.
